(19) United States
(12) Patent Application Publication
(10) Pub. No.: US 2004/0072783 A1
(43) Pub. Date: Apr. 15, 2004

(54) Nucleozymes with endonuclease activity

(76) Inventors: Ronald Breaker, Guilford, CT (US); Gail Emilsson, Wallingford, CT (US)

Correspondence Address: Michael S. Greenfield, McDonnell Boehnen Hulbert & Berghoff, 32nd Floor, 300 S. Wacker Drive, Chicago, IL, 60606, US

(21) Appl. No.: 10/457,039

(22) Filed: Jun. 6, 2003

Related U.S. Application Data
(63) Continuation of application No. 09/781,902, filed on Feb. 8, 2001, now abandoned.
(60) Provisional application No. 60/193,646, filed on Mar. 31, 2000.

(51) Int. Cl. A61K 48/00; C07H 21/04

(52) U.S. Cl. 514/44; 536/23.2

(57) ABSTRACT

The present invention relates to nucleozymes (DNA catalysts, DNA enzymes), methods of synthesis, and uses thereof.

This patent application claims priority from Breaker et al., U.S. S No. (60/193,646), filed Mar. 31, 2000, entitled “NUCLEOZYMES WITH ENDONUCLEASE ACTIVITY”. This patent application is hereby incorporated by reference herein in its entirety including the drawings.

### BACKGROUND OF THE INVENTION

This invention relates to nucleozymes, specifically deoxyribonucleic acid molecules, with catalytic activity and derivatives thereof.

The following is a brief description of enzymatic nucleic acid molecules. This summary is not meant to be complete but is provided only for understanding of the invention that follows. This summary is not an admission that all of the work described below is prior art to the claimed invention.

Enzymatic nucleic acid molecules are nucleic acid molecules capable of catalyzing one or more of a variety of reactions, including the ability to repeatedly cleave (multiple turnover) other separate nucleic acid molecules in a nucleotide base sequence-specific manner. Such enzymatic nucleic acid molecules can be used, for example, to target virtually any RNA transcript (Zaug et al., 324, Nature 429 1986; Cech, 260 JAMA 3030, 1988).

Because of their sequence-specificity, trans-cleaving enzymatic nucleic acid molecules show promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the mRNA non-functional and abrogates protein expression from that RNA. In this manner, synthesis of a protein associated with a disease state can be selectively inhibited.

In general, enzymatic nucleic acids with RNA cleaving activity act by first binding to a target RNA. Such binding occurs through the target-binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches have been used to evolve new nucleic acid catalysts, for example, in vitro selection and/or in vitro evolution strategies (Orgel, 1979, Proc. R. Soc. London, B 205, 435). These approaches have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al. 1992, Science 257, 635-641; Joyce, 1992, Scientific American 267, 90-97; Breaker et al. 1994, TIBTECH 12, 268; Bartel et al., 1993, Science 261:1411-1418; Szostak, 1993, TIBS 17, 89-93; Kumar et al. 1995, FASEB J., 9, 1183; Breaker, 1996, Curr. Op. Biotech., 7, 442).

Catalytic nucleic acid molecules until recently were all known to require the presence of 2′-hydroxyl (2′-OH) groups within the molecule for their enzymatic function. Usman and co-workers recently reported that nucleic acid molecules lacking a 2′-hydroxyl group, such as DNA molecules, can catalyze a chemical reaction (Chartrand et al., 1995, Nucleic Acids Research, 23(20), 4092-4096; Usman et al., U.S. Pat. No. 5,861,288). There have also been several reports of non-2′-OH containing nucleic acid molecules that are capable of catalyzing chemical reactions (Li and Breaker, 1999, Crit. Opin. Struct. Biol., 9, 315-323; Breaker, 1999, Nature Biotechnology, 17, 422-423). The use of in vitro selection and/or in vitro evolution techniques applied to random pools of single-stranded DNA has provided catalytic DNA, or deoxyribozymes, capable of catalyzing the cleavage of RNA (Usman et al., U.S. Pat. No. 5,861,288; Breaker and Joyce, 1994, Chem. Biol., 1, 223-229; Breaker and Joyce, 1995, Chem. Biol., 655-660), facilitation of the ligation of chemically activated DNA (Cuenoud and Szostak, 1995, Nature, 375, 611-614), and the metallation of porphyrin rings (Li and Sen, 1996, Nat. Struct. Biol., 3, 743-747).

The cleavage of RNA by transesterification via catalytic DNA structures is often dependant on metal ion cofactors, such as Mg²⁺, Mn²⁺, Zn²⁺, Pb²⁺, Ca²⁺, and Cd²⁺, or an amino acid cofactor such as histidine (Roth and Breaker, 1998, PNAS USA., 95, 6027-6031). However, the native structure of DNA alone may be sufficient in providing the chemical groups that are responsible for catalysis (Geyer and Sen. 1997, Chem. Biol., 4, 579-593).

Joyce and Breaker, U.S. Pat. No. 5,807,718, describe specific magnesium dependent RNA cleaving DNA enzymes with defined structural and target sequence constraints.

### SUMMARY OF THE INVENTION

This invention relates to nucleic acid molecules with catalytic activity, which are particularly useful for cleavage of RNA or DNA. The nucleic acid catalysts of the instant invention are distinct from other nucleic acid catalysts known in the art. The nucleic acid catalysts of the instant invention do not share sequence homology with other known DNA enzymes. Specifically, the nucleic acid catalysts of the instant invention are capable of catalyzing an intermolecular or intramolecular endonuclease reaction.

In a preferred embodiment, the invention features a nucleic acid molecule with catalytic activity having either the formulae I or II:

3′-X-Z-Y-5′ Formula I

3′-X-W-V-R-Y-5′ Formula II

In the above formulae, X and Y are independently oligonucleotides of length sufficient to stably interact (e.g., by forming hydrogen bonds with complementary nucleotides in the target) with a target nucleic acid molecule (the target can be an RNA, DNA or RNA/DNA mixed polymers, including polymers that may include base, sugar, and/or phosphate nucleotide modifications; such modifications are preferably naturally occurring modifications), preferably, the length of X and Y are independently between 3-20 nucleotides long, e.g., specifically, 3, 4, 5, 6, 7, 8, 9, 10, 11, 12, 13, 14, 15, 17, and 20); X and Y may have the same lengths or may have different lengths; —represents a chemical linkage (e.g. a phosphate ester linkage, amide linkage or others known in the art); Z is independently a nucleotide sequence comprising 5′-GATGCAGCTGGGGAGGCGTTT-3′ (SEQ ID NO 51), R is independently a nucleotide sequence comprising 5′-GGGGA-3′, V represents a nucleotide or non-nucleotide linker, which may be present or absent, (i.e., the molecule is assembled from two separate molecules), but when present, is a nucleotide and/or non-nucleotide linker, which may comprise a single-stranded and/or double-stranded region; W is independently a nucleotide sequence selected from the group comprising 5′-TGGGGAAGCACAGGGT-3′ (SEQ ID NO 52), 5′-TGGGGAAGCTCTGGGT-3′ (SEQ ID NO 53). 5′-TGGGGAAGCACAGGGT-3′ (SEQ ID NO 54), and 5′-TGGGGAAGCACATGGT-3′ (SEQ ID NO 55); additions, deletions, and substitutions to these sequences may be made without significantly altering the activity of the molecules and are hence within the scope of the invention; and C, G, A, and T represent cytidine, guanosine, adenosine and thymidine nucleotides, respectively. The nucleotides in each of the formulae I and II are unmodified or modified at the sugar, base, and/or phosphate as known in the art.

In a preferred embodiment, the nucleotide linker (V) is a nucleic acid sequence selected from the group consisting of 3′-ACCTGAGGG-3′,5′-GCGTTAG-3′ and 5′-AGGAAGCATCTTATGCGACC-3′ (SEQ ID NO 56).

The nucleotide linker V is preferably 5-40 nucleotides in length, more preferably 7-20 nucleotides in length and still more preferably 7-12 nucleotides in length.

In yet another embodiment, the nucleotide linker (V) is a nucleic acid aptamer, such as an ATP aptamer, HIV Rev aptamer (RRE), HIV Tat aptamer (TAR) and others (for a review, see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The ligand can be any natural or synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In another embodiment, the non-nucleotide linker (V) is as defined herein. The term “non-nucleotide” as used herein includes either abasic nucleotide, polyether, polyamine, polyamide, peptide, carbohydrate, lipid, or polyhydrocarbon compounds. These compounds can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenine, guanine, cytosine, uracil or thymine. The terms “abasic” or “abasic nucleotide” as used herein encompass sugar moieties lacking a base or having other chemical groups in place of a nucleotide base at the 1′ position. Specific examples of non-nucleotides include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al. Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. Thus, in a preferred embodiment, the invention features an enzymatic nucleic acid molecule having one or more non-nucleotide moieties, and having enzymatic activity to cleave an RNA or DNA molecule.

By ‘nucleozyme’ or ‘DNA enzyme’ or ‘DNAzyme’ or “deoxyribozyme” as used herein is meant, an enzymatic nucleic acid molecule that does not require the presence of a ribonucleotide (2′-OH) group in the molecule for its activity. These molecules are also referred to as catalytic DNA, nucleic acid catalysts, restriction endonucleases, catalytic oligonucleotides, and enzymatic DNA molecules. In particular embodiments, the enzymatic nucleic acid molecule may have an attached linker(s) or other attached or associated groups, moieties, or chains containing one or more nucleotides with 2′-OH groups. DNAzymes can be synthesized chemically or expressed endogenously in vivo, by means of a single stranded DNA vector or equivalent thereof.

By “enzymatic nucleic acid molecule” it is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% may also be useful in this invention (see for example Werner and Uhlenbeck, 1995, Nucleic Acids Research, 23, 1092-2096; Hammann et al., 1999, Antisense and Nucleic Acid Drug Dev., 9, 25-31). The nucleic acids may be modified at the base, sugar, and/or phosphate groups.

The enzymatic nucleic acid molecule (e.g., the molecules of formulae I and II) of the instant invention are capable of catalyzing (altering the velocity and/or rate of) a variety of reactions including the ability to repeatedly cleave (multiple turnover) other separate nucleic acid molecules (endonuclease activity) in a nucleotide base sequence-specific manner. Such a molecule with endonuclease activity can have complementarity in a substrate binding region (e.g. X and Y in formulae I and II) to a specified gene target, and also has an enzymatic activity that specifically cleaves RNA or DNA in that target. That is, the nucleic acid molecule with endonuclease activity is able to intramolecularly or intermolecularly cleave RNA or DNA and thereby inactivate a target RNA or DNA molecule. This complementarity functions to allow sufficient hybridization of the enzymatic DNA molecule to the target RNA or DNA to allow the cleavage to occur. 100% complementarity is preferred, but complementarity as low as 50-75% may also be useful in this invention. The nucleic acids can be modified at the base, sugar, and/or phosphate groups. All that is required, as will be readily recognized by persons skilled in the art, is that the enzymatic nucleic acid molecule not be dependent on the presence of a ribonucleotide in the molecule for its catalytic activity.

In preferred embodiments, the enzymatic nucleic acid includes one or more stretches of non-ribonucleotide containing oligonucleotide, which provide the enzymatic activity of the molecule. The necessary non-ribonucleotide components are known in the art.

Thus, in one preferred embodiment, the invention features DNA enzymes that inhibit gene expression and/or cell proliferation. These chemically or enzymatically synthesized nucleic acid molecules contain substrate binding domains that bind to accessible regions of specific target nucleic acid molecules. The nucleic acid molecules also contain domains that catalyze the cleavage of target. Upon binding, the enzymatic nucleic acid molecules cleave the target molecules, preventing, for example, translation and protein accumulation. In the absence of the expression of the target gene, cell proliferation, for example, is inhibited. In another aspect of the invention, enzymatic nucleic acid molecules that cleave target molecules are expressed from a single stranded DNA intracellular expression vector. Preferably, the vectors capable of expressing the DNA enzymes are delivered as described below, and persist in target cells. Suitable vectors can be used that provide for transient expression of DNA enzymes. Such vectors can be repeatedly administered as necessary. Once expressed, the DNA enzymes cleave the target mRNA. Delivery of DNA enzyme expressing vectors can be systemic, such as by intravenous or intramuscular administration, by administration to target cells ex-planted from the patient followed by reintroduction into the patient, or by any other means that would allow for introduction into the desired target cell (for a review, see Couture and Stinchcomb, 1996, TIG., 12, 510).

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., cleavage via a DNAzyme. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “sufficient length” is meant an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. Preferably, the binding arms are not so long as to prevent useful turnover of the nucleic acid molecule.

By “stably interact” is meant interaction of the oligonucleotides with target nucleic acid (e.g., by forming hydrogen bonds with complementary nucleotides in the target under physiological conditions) that is sufficient to the intended purpose (e.g., cleavage of target RNA by an enzyme).

By “inhibit” it is meant that the activity of a given protein or level of RNAs or equivalent RNAs encoding one or more protein subunits of a given protein target is reduced below that observed in the absence of the nucleic acid molecules of the invention. In one embodiment, inhibition with enzymatic nucleic acid molecule preferably is below that level observed in the presence of an enzymatically inactive or attenuated molecule that is able to bind to the same site on the target RNA, but is unable to cleave that RNA. In another embodiment, inhibition of target genes with the nucleic acid molecule of the instant invention is greater than in the presence of the nucleic acid molecule than in its absence.

By “nucleic acid molecule” as used herein is meant a molecule having nucleotides. The nucleic acid can be single, double, or multiple stranded and may comprise modified or unmodified nucleotides or non-nucleotides or various mixtures and combinations thereof.

By “gene” it is meant a nucleic acid that encodes an RNA, for example, nucleic acid sequences including but not limited to structural genes encoding a polypeptide.

By “RNA” is meant a molecule comprising at least one ribonucleotide residue. By “ribonucleotide” is meant a nucleotide with a hydroxyl group at the 2′ position of a β-D-ribo-furanose moiety.

As used in herein “cell” is used in its usual biological sense, and does not refer to an entire multicellular organism, e.g., specifically does not refer to a human. The cell may be present in an organism which may be a human but is preferably a non-human multicellular organism, e.g., birds, plants and mammals such as cows, sheep, apes, monkeys, swine, dogs, and cats. The cell may be prokaryotic (e.g., bacterial cell) or eukaryotic (e.g., mammalian or plant cell).

By “patient” is meant an organism which is a donor or recipient of explanted cells or the cells themselves. “Patient” also refers to an organism to which enzymatic nucleic acid molecules can be administered. Preferably, a patient is a mammal or mammalian cells. More preferably, a patient is a human or human cells.

By “vectors” is meant any nucleic acid- and/or viral-based technique used to deliver a desired nucleic acid.

In another preferred embodiment, catalytic activity of the molecules described in the instant invention can be optimized. Modifications which enhance their efficacy in cells, and removal of bases from stem loop structures to shorten DNA synthesis times and reduce chemical requirements are desired. Catalytic activity of the molecules described in the instant invention can be optimized as described by Usman et al., U.S. Pat. No. 5,861,288. The details will not be repeated here, but include altering the length of the DNA enzyme binding arms, or chemically synthesizing DNA enzymes with modifications (base, sugar and/or phosphate) that prevent their degradation by serum nucleases and or enhance their enzymatic activity (see e.g. Eckstein et al., International Publication No. WO 92/07065; Perrault et al., 1990 Nature 344, 565; Pieken et al., 1991 Science 253, 314; Usman and Cedergren, 1992 Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra: all of these describe various chemical modifications that can be made to the base, phosphate and/or sugar moieties of enzymatic nucleic acid molecules). All these publications are hereby incorporated by reference herein.

By “enhanced enzymatic activity” is meant to include activity measured in cells and/or in vivo where the activity is a reflection of both catalytic activity and DNA enzyme stability.

In yet another preferred embodiment, nucleic acid catalysts having chemical modifications which maintain or enhance enzymatic activity are provided. Such nucleic acid is also generally more resistant to nucleases than unmodified nucleic acid. Thus, in a cell and/or in viva the activity may not be significantly lowered. As exemplified herein such enzymes are useful in a cell and/or in vivo even if activity over all is reduced 10-fold (Burgin et al., 1996, Biochemistry, 35, 14090).

In a preferred embodiment, the invention provides a method for producing a class of deoxyribonucleic acid-based gene inhibiting agents which exhibit a high degree of specificity for the RNA of a desired target. For example, the enzymatic nucleic acid molecule is preferably targeted to a highly conserved sequence region of target RNAs encoding target proteins such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., DNA enzymes) can be expressed from single stranded DNA expression vectors that are delivered to specific cells.

By “highly conserved sequence region” is meant, a nucleotide sequence of one or more regions in a target gene does not vary significantly from one generation to the other or from one biological system to the other.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo or in vivo through injection, infusion pump or stent, with or without their incorporation in biopolymers.

In another aspect, the invention provides mammalian cells containing one or more nucleic acid molecules and/or expression vectors of this invention. The one or more nucleic acid molecules may independently be targeted to the same or different sites.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

Other features and advantages of the invention will be apparent from the following description of the preferred embodiments thereof, and from the claims.

### DESCRIPTION OF THE PREFERRED EMBODIMENTS

The drawings will first briefly be described.

### DRAWINGS

FIG. 1 is a diagram that shows the construct on which the DNA enzyme selection is based. Capital letters indicate the four standard DNA bases and RNA bases (reverse type) in regions of the molecular library which were constant among all molecules. These regions provided the RNA substrate, positioned the random region adjacent to the substrate by base-pairing and allowed manipulation of the library during selection using standard methods. Catalytic sequences were derived from the region of 50 nucleotides, imbedded between the constant regions synthesized to contain random DNA sequences. Molecules were selected on the basis of their ability to self-cleave at an RNA residue. Molecules were reacted in solution in a buffer which approximated the composition of Escherichia coli cytoplasm with respect to conditions expected to be relevant to RNA cleavage

FIG. 2 is a diagram that shows a non-limiting secondary-structure model for Clone 27 of a class I DNA enzyme of the instant invention. The secondary structure shown in FIG. 2a is based on the region of conserved nucleotides underlined in Table 2 with base pairing restored at the 3′ end of the catalyst. The K_{d} for this enzyme interacting with substrate is 5 μM. The enzyme does not require glutathione but does lose 10-fold activity in the absence of putrescine. Its pKₐ is ˜7.9, reaching a maximum pseudo first order rate constant in 0.5 mM Mg²⁺ of 0.06 min⁻¹. The enzyme also cleaves the in vitro transcribed target shown in FIG. 2b, demonstrating that it does not require DNA residues in the substrate and can be generalized by changing the substrate binding arms.

FIG. 3 is a diagram showing a non-limiting secondary structure model for clone 37 of a class II DNA enzyme of the instant invention.

FIG. 4 is a diagram that shows a selection scheme for the isolation of self-cleaving DNA enzymes from a random-sequence DNA population.

FIG. 5 is a diagram that shows the substrate sequence requirements of Class I motif nucleozymes of the invention.

FIG. 6 is a diagram that shows the kinetic parameters of a Class I motif nucleozyme of the invention.

FIG. 7 is a diagram showing proposed secondary structures of Class IV and Class V trans-cleaving nucleozymes of the invention.

Nucleic Acid Catalysts

The invention provides nucleic acid catalysts and methods for producing a class of enzymatic nucleic acid cleaving agents which exhibit a high degree of specificity for the nucleic acid sequence of a desired target. The enzymatic nucleic acid molecule is preferably targeted to a highly conserved sequence region of a target such that specific diagnosis and/or treatment of a disease or condition in a variety of biological systems can be provided with a single enzymatic nucleic acid. Such enzymatic nucleic acid molecules can be delivered exogenously to specific cells as required. In the preferred Class I and II motifs, the small size (less than 60 nucleotides, preferably between 25-40 nucleotides in length) of the molecule allows the cost of treatment to be reduced.

In preferred embodiments of the present invention, a nucleic acid molecule, e.g., an enzymatic deoxyribonucleic acid molecule, is 13 to 100 nucleotides in length, e.g., in specific embodiments 35, 36, 37, or 38 nucleotides in length (e.g., for particular DNA enzymes). In particular embodiments, the nucleic acid molecule is 15-100, 17-100, 20-100, 21-100, 23-100, 25-100, 27-100, 30-100, 32-100, 35-100, 40-100, 50-100, 60-100, 70-100, or 80-100 nucleotides in length. Instead of 100 nucleotides being the upper limit on the length ranges specified above, the upper limit of the length range can be, for example, 30, 40, 50, 60, 70, or 80 nucleotides. Thus, for any of the length ranges, the length range for particular embodiments has lower limit as specified, with an upper limit as specified which is greater than the lower limit. For example, in a particular embodiment, the length range can be 35-50 nucleotides in length. All such ranges are expressly included. Also in particular embodiments, a nucleic acid molecule can have a length which is any of the lengths specified above, for example, a catalytic core of 21 conserved nucleotides in length with variable length binding arms and/or variable regions.

The enzymatic nucleic acid molecules of Formulae I and II may independently comprise a cap stricture which may independently be present or absent.

By “chimeric nucleic acid molecule” or “mixed polymer” is meant that, the molecule may be comprised of both modified or unmodified nucleotides.

By “cap structure” is meant chemical modifications, which have been incorporated at either terminus of the oligonucleotide. These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both termini. In non-limiting examples the 5′-cap is selected from the group consisting of the following: inverted abasic residue (moiety); 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′ phosphate; 3′-phosphorothioate; phosphorodithioate; or bridging or non-bridging methylphosphonate moiety (for more details, see Beigelman et al., International PCT publication No. WO 97/26270, incorporated by reference herein).

In yet another preferred embodiment, the 3′-cap is selected from a group consisting of the following: 4′,5′-methylene nucleotide; 1-(beta-D-erythrofuranosyl) nucleotide; 4′-thio nucleotide; carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide; 5′-5′-inverted nucleotide moiety; 5′-5′-inverted abasic moiety; 5′-phosphoramidate; 5′-phosphorothioate; 1,4-butanediol phosphate; 5′-amino: bridging and/or non-bridging 5′-phosphoramidate; phosphorothioate and/or phosphorodithioate; bridging or non bridging methylphosphonate; and 5′-mercapto moieties (for more details see Beaucage and Iyer, 1993, Tetrahedron 49, 1925; incorporated by reference herein).

By “oligonucleotide” as used herein, is meant a molecule comprising two or more nucleotides.

The specific enzymatic nucleic acid molecules described in the instant application are not limiting in the invention and those skilled in the art will recognize that all that is important in an enzymatic nucleic acid molecule of this invention is that it has a specific substrate binding site (e.g., X and Y of Formulae I and II above) which is complementary to one or more of the target nucleic acid regions, and that it have nucleotide sequences within or surrounding that substrate binding site which impart a nucleic acid cleaving activity to the molecule.

The determination of whether the V region can be deleted or changed can be determined by experimentation and tested in vitro using the methods described herein. Similarly, when the V region is present the determination or whether its length can be increased or decreased can be evaluated using the selection protocols described herein.

By “enzymatic portion” is meant that part of the DNA enzyme essential for cleavage of an RNA substrate.

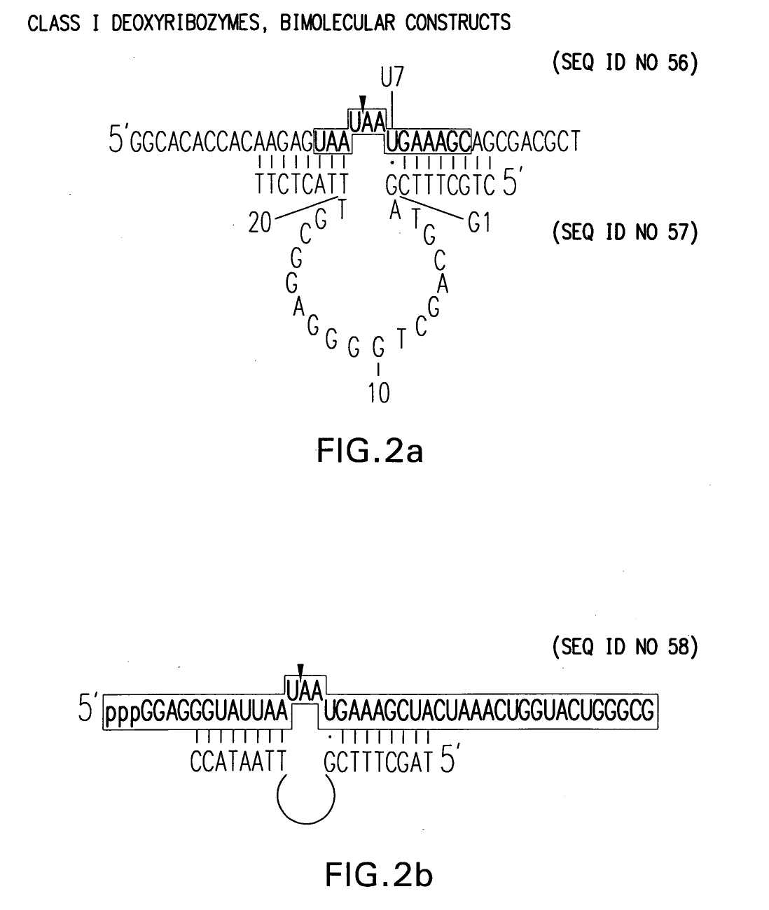

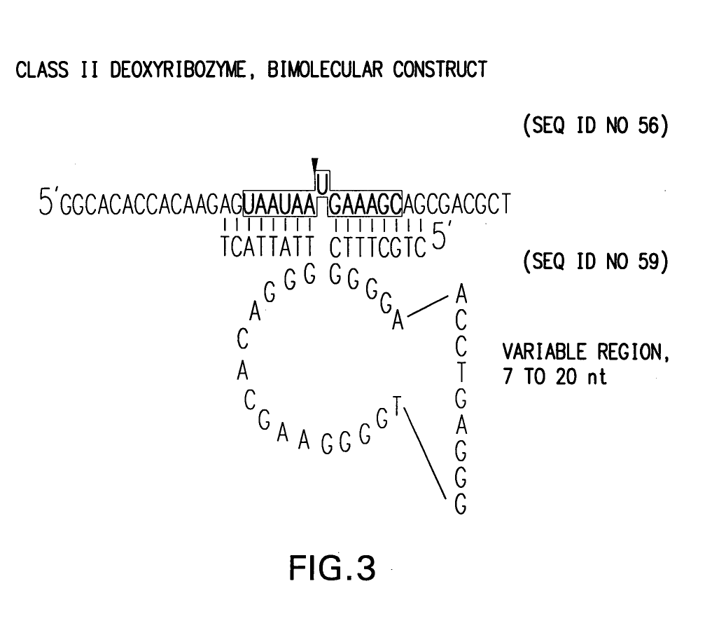

By “substrate binding arm” or “substrate binding domain” is meant that portion of a DNAzyme which is complementary to (i.e., able to base-pair with) a portion of its substrate. Generally, such complementarity is 100%, but can be less if desired. For example, as few as 10 nucleotides out of a total of 14 may be base-paired. Such arms are shown generally in FIGS. 2 and 3 and as X and Y in Formulae I and II. That is, these arms contain sequences within a DNA enzyme which are intended to bring DNAzyme and target RNA together through complementary base-pairing interactions. The DNAzymes of the invention can have binding arms that are contiguous or non-contiguous and can be of varying lengths. The length of the binding arm(s) are preferably greater than or equal to four nucleotides; specifically 12-100 nucleotides; more specifically 14-24 nucleotides long. The two binding arms are chosen, such that the length of the binding arms are symmetrical (i.e., each of the binding arms is of the same length; e.g., five and five nucleotides, six and six nucleotides or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

Catalytic activity of the DNA enzymes described in the instant invention can be optimized as described by Usman et al., U.S. Pat. No. 5,807,718. The methods described by Draper et al., supra for nucleic acid catalysts can readily be applied for use in the optimization of the nucleic acid molecules of the instant invention. Specific details will not be repeated here, but include altering the length of the DNAzyme binding arms, or chemically synthesizing DNAzymes with modifications (base, sugar and/or phosphate) that prevent their degradation by serum ribonucleases and/or enhance their enzymatic activity (see e.g., Eckstein et al., International Publication No. WO 92/07065; Perrault et al., 1990 Nature 344, 565; Pieken et al., 1991 Science 253, 314; Usman and Cedergren, 1992 Trends in Biochem. Sci. 17, 334; Usman et al., International Publication No. WO 93/15187; Rossi et al., International Publication No. WO 91/03162; Sproat, U.S. Pat. No. 5,334,711; and Burgin et al., supra; all of these describe various chemical modifications that can be made to the base, phosphate and/or sugar moieties of enzymatic DNA and/or RNA molecules). All these publications are hereby incorporated by reference herein. Modifications which enhance their efficacy in cells, and removal of bases from stem loop structures to shorten DNA synthesis times and reduce chemical requirements are desired. The enzymatic nucleic acid molecules can be synthesized entirely of deoxyribonucleotides, or other 2′-modified nucleotides (e.g.; 2′-O-methyl, 2′-O-allyl, 2′-C-allyl, 2′-deoxy-2′-amino, 2′-deoxy-2′-fluoro, 2′-O-amino etc.), individually or in combination, so long as the nucleic acid catalyst is functional.

Therapeutic DNAzymes should remain stable within cells until translation of the target RNA has been inhibited long enough to reduce the levels of the undesirable protein. This period of time varies between hours to days depending upon the disease state. In particular, DNAzymes should be resistant to nucleases in order to function as effective intracellular therapeutic agents. Improvements in the chemical synthesis of DNA and RNA (Wincott et al., 1995 Nucleic Acids Res. 23, 2677; incorporated by reference herein) have expanded the ability to modify DNA enzymes to enhance their nuclease stability.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N-6-isopentenyladenosine, ?-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In a preferred embodiment, the invention features modified DNAzymes with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39.

In a further preferred embodiment of the instant invention, an inverted deoxy abasic moiety is utilized at the 3′ end of the enzymatic nucleic acid molecule.

In particular, the invention features modified enzymatic nucleic acid molecules having a base substitution selected from pyridin-4-one, pyridin-2-one, phenyl, pseudouracil, 2,4,6-trimethoxy benzene, 3-methyluracil, dihydrouracil, naphthyl, 6-methyl-uracil and aminophenyl.

There are several examples in the art describing sugar and phosphate modifications that can be introduced into enzymatic nucleic acid molecules without significantly effecting catalysis and with significant enhancement in their nuclease stability and efficacy. Enzymatic nucleic acid molecules are modified to enhance stability and/or enhance catalytic activity by modification with nuclease resistant groups, for example, 2′-amino, 2′-C-allyl, 2′-flouro, 2′-O-methyl, nucleotide base modifications (for a review see Usman and Cedergren, 1992 TIBS 17, 34; Usman et al., 1994 Nucleic Acids Symp. Ser. 31, 163; Burgin et al., 1996 Biochemistry 35, 14090). Sugar modifications of enzymatic nucleic acid molecules have been extensively described in the art (see Eckstein et al., International Publication PCT No. WO 92/07065; Perrault et al. Nature 1990, 344, 565-568; Pieken et al. Science 1991, 253, 314-317; Usman and Cedergren, Trends in Biochem. Sci. 1992, 17, 334-339; Usman et al. International Publication PCT No. WO 93/15187; Sproat, U.S. Pat. No. 5,334,711 and Beigelman et al., 1995 J. Biol. Chem. 270, 25702).

Additionally, the enzymatic nucleic acid molecules can be linked to various chemical moieties and/or ligands to enhance stability, localization, and/or efficacy. Such moieties and ligands include but are not limited to polyethylene glycol (PEG), cholesterol, cytofectins (such as DOPE, DDAB, DOGS, DOTMA and DOTMA analogues including DOTAP, DMRIE, DOSPA, DORIE, DORI, and GAP-DLRIE), glucose, galactose, spermine, spermidine, C-dextran, polyacrylamide, biotin, retinoic acid, peptide nucleic acids, antigens (such as CD40, CD44, carcinoembryonic, endoglin, and prostate-specific antigens), receptors (such as VEGF, HER2/neu), and other fatty acids, steroids, cationic lipids, polyamines, polyamides, glucocorticoids, integrins, histones, protamines, toxins, viroids, virusoids, amino acids, peptides, proteins, sugars, polysaccharides, glycoconjugates, oligonucleotides, metals, small molecules, macromolecules and combinations thereof. For a review of non-limiting carbohydrate modifications including 2′-conjugates, see Manoharan, 1999, Biochem. Biophys. Acta., 1489(1), 117-130. For a review of non-limiting drug macromolecule conjugates, see Takakura et al., 1996, Advan. Drug, Delivery Rev., 19(3), 377-399. Such publications describe general methods and strategies to determine the location of incorporation of sugar, base and/or phosphate modifications and the like into nucleic acid molecules without inhibiting catalysis, and are incorporated by reference herein. In view of such teachings, similar modifications can be used as described herein to modify the nucleic acid catalysts of the instant invention.

As the term is used in this application, non-nucleotide-containing enzymatic nucleic acid means a nucleic acid molecule that contains at least one non-nucleotide component which replaces a portion of a DNAzyme, e.g., but not limited to, a double-stranded stem, a single-stranded “catalytic core” sequence, a single-stranded loop or a single-stranded recognition sequence. These molecules are able to cleave (preferably, repeatedly cleave through multiple turnover) separate RNA or DNA molecules in a nucleotide base sequence specific manner. Such molecules can also act to cleave intramolecularly if that is desired. Such enzymatic molecules can be targeted to virtually any RNA transcript.

The sequences of DNAzymes that are chemically synthesized, useful in this invention, are shown in the Tables and Figures. Those in the art will recognize that these sequences are representative only of many more such sequences where the enzymatic portion of the enzymatic nucleic acid molecule (all but the binding arms) is altered to affect activity. The DNAzyme sequences listed in the tables and figures may be formed of deoxyribonucleotides or other nucleotides or non-nucleotides. Such DNAzymes with enzymatic activity are equivalent to the exemplary DNAzymes described herein with specificity in the tables and figures.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs no more than 100 nucleotides in length, preferably no more than 80 nucleotides in length, and most preferably no more than 50 nucleotides in length; e.g., DNA enzymes) are preferably used for exogenous deliver. The simple structure of these molecules increases the ability of the nucleic acid to invade targeted regions of RNA structure. Exemplary molecules of the instant invention are chemically synthesized, and others can similarly be synthesized.

Oligonucleotides (e.g.; DNAzymes) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table 1 outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™) Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder.

The method of synthesis used for normal RNA follows the procedure as described in Usman et al., 1987, J. Am. Chem. Soc. 109, 7845; Scaringe et al., 1990, Nucleic Acids Res., 18, 5433; Wincott et al., 1995, Nucleic Acids Res. 23, 2677-2684 and Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table 1 outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical. Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA•3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine DMSO: 1/1 (0.8 mL) at 65° C. for 15 min. The vial is brought to room temperature TEA•3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the examples described above including but not limited to 96-well format, all that is important is the ratio of chemicals used in the reaction.

Alternatively, the nucleic acid molecules of the present invention can be synthesized separately and joined together post-synthetically, for example, by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, (for a review, see Usman and Cedergren, 1992, TIBS 17, 34; Usman et al., 1994, Nucleic Acids Symp. Ser. 31, 163). DNAzymes are purified by gel electrophoresis using general methods or are purified by high pressure liquid chromatography (HPLC; see Wincott et al., supra, the totality of which is hereby incorporated herein by reference) and are re-suspended in water.

Administration of DNAzymes

Sullivan et al., PCT WO 94/02595, describe the general methods for delivery of enzymatic nucleic acid molecules. DNAzymes can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, DNAzymes may be directly delivered ex vivo to cells or tissues with or without the aforementioned vehicles. Alternatively, the DNAzymes/vehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of nucleic acid molecule delivery and administration are provided in Sullivan et al., supra and Draper et al., PCT WO93/23569 which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration, suppositories for rectal administration, sterile solutions, suspensions for injectable administration, and other compositions known in the art.

The present invention also includes pharmaceutically acceptable formulations of the compounds described. These formulations include salts of the above compounds, e.g. acid addition salts, including salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example, oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

The invention also features the use of a composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. Such liposomes have been shown to accumulate selectively in tumors, presumably by extravasation and capture in the neovascularized target tissues (Lasic et al., Science 1995, 267, 1275-1276; Oku et al, 1995, Biochim. Biophys. Acta, 1238, 86-90).

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be added to the compositions. Id. at 1449. Suitable examples include sodium benzoate, sorbic acid and esters of p-hydroxybenzoic acid. In addition, antioxidants and suspending agents can be added to the compositions.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer. In a one aspect, the invention provides enzymatic nucleic acid molecules that can be delivered exogenously to specific cells as required. The enzymatic nucleic acid molecules are added directly, or can be complexed with cationic lipids, packaged within liposomes, or otherwise delivered to smooth muscle cells. The RNA or RNA complexes can be locally administered to relevant tissues through the use of a catheter, infusion pump or stent, with or without their incorporation in biopolymers. Using the methods described herein, other enzymatic nucleic acid molecules that cleave target nucleic acid may be derived and used as described above. Specific examples of nucleic acid catalysts of the instant invention are provided below in the Tables and Figures.

Alternatively, the enzymatic nucleic acid molecules of the instant invention can be expressed from single stranded DNA expression vectors.

The invention also features a method for enhancing the effect of the nucleic acid catalyst of the instant invention in vivo. The method includes the step of causing the nucleic acid catalyst to be localized in vivo with its target. In a related aspect, the invention features nucleic acid catalysts which are adapted for localization with the viral target of the agent in vivo.

Those in the art will recognize that many methods can be used for modification of nucleic acid catalyst such that they are caused to be localized in an appropriate compartment with a target. Examples of these methods follow but are not limiting in the invention. Thus, for example, the nucleic acid catalysts of the invention can be synthesized in vivo from vectors (or formed in vitro) such that they are covalently or noncovalently bonded with a targeting agent, examples of which are well known in the art (Sullenger et al., U.S. Pat. No. 5,854,038; Castanotto et al., Methods Enzymol 2000:313:401-20; Rossi et al., Science 1999, 285,1685). These targeting agents are termed “localization signals”. In addition, nucleic acid catalysts can be synthesized in vitro and administered in any one of many standard methods to cause the nucleic acid catalysts to be targeted to an appropriate cellular compartment within a patient.

By “enhancing” the effect of a nucleic acid catalysts in vivo is meant that a localization signal targets that nucleic acid catalysts to a specific site within a cell and thereby causes that nucleic acid catalysts to act more efficiently. Thus, a lower concentration of nucleic acid catalysts administered to a cell in vivo can have an equal effect to a larger concentration of non-localized nucleic acid catalysts. Such increased efficiency of the targeted or localized nucleic acid catalysts can be measured by any standard procedure well-known to those of ordinary skill in the art. In general, the effect of the nucleic acid catalyst is enhanced by placing the nucleic acid catalyst in a closer proximity with the target, so that it may have its desired effect on that target. This may be achieved by causing the nucleic acid catalysts to be located in a small defined compartment with the target (e.g., within a viral particle), or to be located in the same space within a compartment, e.g., in a nucleus at the location of synthesis of the target.

Localization signals include any proteinaceous or nucleic acid component which naturally becomes localized in the desired compartment, for example, a viral packaging signal, or its equivalent. Localization signals can be identified by those in the art as those signals which cause the nucleic acid catalysts to which they are associated with to become localized in certain compartments, and can be readily discovered using standard methodology (Sullenger et al., U.S. Pat. No. 5,854,038; Shaji et al, U.S. Pat. No. 5,834,186). These localization signals can be tethered to the nucleic acid catalysts by any desired procedure, for example, by construction of a DNA template which produces both the localization signal and nucleic acid catalysts as part of the same molecule, or by covalent or ionic bond formation between two moieties. All that is essential in the invention is that the nucleic acid catalysts be able to have its inhibitory effect when localized in the target site, and that the localization signal be able to localize that nucleic acid catalysts to that target site. For example, localization signals such as HIV's Rev response element can be linked to the DNA enzyme to sort in some unique way. Those skilled in the art will recognize that other nucleic acid localization elements may be attached to the DNAzymes of the instant invention using methods known in the art.

Other examples include any cellular RNA/DNA localization signal which causes RNA/DNA containing the signal to be sorted into a pathway which does not contain large numbers of incorrect targets; viral protein localization/assembly signals, e.g., Rev or gag proteins.

Increasing the concentration of a viral inhibitor at an intracellular site important for viral replication or assembly is a general way to increase the effectiveness of nucleic acid catalysts. The above-described co-localization strategy can make use of, for example, a viral packaging signal to co-localize nucleic acid catalysts with a target responsible for viral replication. In this way viral replication can be reduced or prevented. This method can be employed to enhance the effectiveness of nucleic acid catalysts by tethering them to an appropriate localization signal to sort them to the therapeutically important intracellular and viral location where the viral replication machinery is active.

Such co-localization or regulation of DNAzyme strategies are not limited to using naturally occurring localization and regulation signals. Nucleic acid catalysts can be targeted to important intracellular locations by use of artificially evolved DNA/RNAs and/or protein decoys (Szostak, 17 TIBS 89, 1992). These evolved molecules are selected, for example, to bind to a viral protein and may be used to co-localize nucleic acid catalysts with a viral target by tethering the inhibitor to such a decoy.

By “consists essentially of” is meant that the active DNAzyme contains an enzymatic center or core equivalent to those in the examples, and binding arms able to bind target nucleic acid molecules such that cleavage at the target site occurs. Other sequences may be present which do not interfere with such cleavage.

The nucleic acid catalyst of the instant invention can be used to inhibit expression of foreign or endogenous genes, in vitro or in vivo, in prokaryotic cells or in eukaryotic cells, in bacteria, fungi, mycoplasma, archebacteria, algae, plants or any other biological system.

By “endogenous” gene is meant a gene normally found in a cell in its natural location in the genome.

By “foreign” or “heterologous” gene is meant a gene not normally found in the host cell, but that is introduced by standard gene transfer techniques or acquired as result of an infection (e.g., bacterial, viral or fungal infection).

By a “plant” is meant a photosynthetic organism, either eukaryotic and prokaryotic.

### EXAMPLES

The following are non-limiting examples showing the selection, isolation, synthesis and activity of enzymatic nucleic acids of the instant invention.

Applicant employed in vitro selection to isolate populations of Mg²⁺-dependent self-cleaving DNA enzymes from a pool of random-sequence molecules. Characterization of a small number of individual DNAzymes from various populations revealed the emergence of at least six classes of DNA enzymes that adopt distinct secondary structure motifs. None of the six classes corresponds to a previously known folding pattern. Each prototypic DNA enzyme promotes self-cleavage with a chemical rate enhancement of at least 1000-fold above the corresponding uncatalyzed rate.

### Example 1

### In Vitro Selection of Catalytic DNAs from a Random Pool

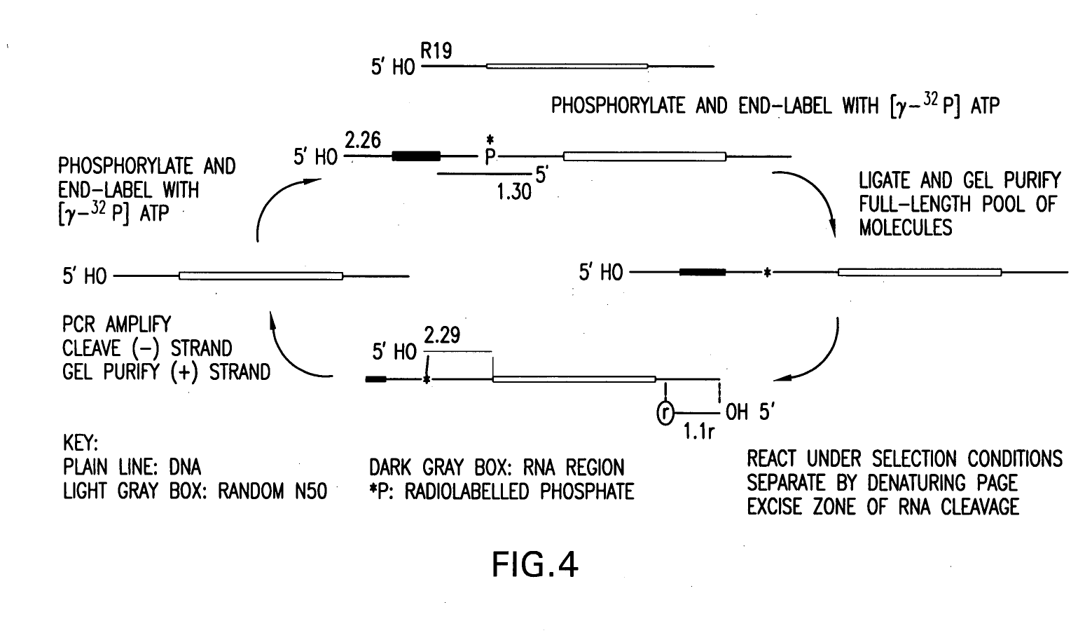

The initial random population was created by ligating a pool of molecules, R19 (5′-ACGTGTGCAGCTTTC[N50]TTATTACGGTAACGTTGGCAC, where N50 indicates the random region of 50 nucleotides) (SEQ ID NO. 56) to a synthetic RNA/DNA chimeric substrate, 2.26(5′-GGCACACCACAAGAGUAAUAAUGAAAGAAGCGACGCT, where underlined type indicates the RNA region) (SEQ ID NO. 57) using T4 DNA ligase and the oligo 1.30 (5′-TGCACACGTAGCGTCGCT) (SEQ ID NO. 56) to template the ligation. The pool of synthetic R19 molecules had first been phosphorylated and end-labelled with [γ-³²P]ATP using T4 polynucleotide kinase to provide the 5′ phosphate necessary for T4 ligase. The full-length population was separated from unligated components by denaturing PAGE. Following elution from the gel, the full-length molecules were ethanol precipitated and resuspended either in 0.001% SDS (early rounds) or directly in 1× selection buffer (later rounds). The population was then reacted at 37° C. in the selection buffer and successful catalysts were separated from unreacted molecules by denaturing PAGE. A zone of gel was excised to encompass all possible RNA cleavage events. Eluted catalysts were amplified by PCR using Taq DNA polymerase and primers 1.1 (5′-GTGCCAACGTTACCG) (SEQ ID NO. 59) and 2.29 (5′-ACGTGTGCAGCTTC) (SEQ ID NO. 60) to create multiple copies of the successful catalysts. A portion of the reaction was then amplified with PCR using primers 1.1r (5′-GTGCCAACGTTACCG, which terminates with a ribose) (SEQ ID NO. 61) and 2.29 to produce molecules with a ribose imbedded in the negative strand. The negative strand was cleaved at this ribose with alkali and heat. The positive strand was then separated from the negative strand fragments by denaturing PAGE. The positive strand was phosphorylated and ligated to the substrate 2.26 as for the initial population. The new pool was then purified from unincorporated substrate and ligation template by denaturing PAGE for the next round of selection. FIG. 4 is a schematic representation of the selection process described above.

Generation 0 composed of the initial random pool was reacted at 37° C. for 12 hours in a buffer that was 50 mM magnesium chloride, 20 mM for amino acids with pKₐ's nearest neutral (arg, asn, cys, gln, glu, his, lys, met, phe, ser), 1 mM for tyrosine (due to its low solubility), and 2 mM for the remaining natural amino acids. The selection buffer also contained, in every round, 150 mM potassium chloride, 7 mM putrescine (to potentially assist in folding), and 10 mM reduced glutathione and was adjusted to pH 7.6±0.1 at 37° C. after addition of all components. Both the reaction time and the concentrations of magnesium and the amino acids were reduced over the course of selection to final values of 10 minutes reaction time and 0.5 mM of both magnesium and each amino acid. When the amino acid content was reduced, HEPES was added at 50 mM to compensate for the loss in buffering.

During the selection process, the times allowed for ligation and for elution of the pool from the ligation acrylamide gel were reduced to prevent RNA cleavage during these processes. Glutathione was included in the selection buffer at a cytoplasm-like concentration to keep the components, especially cysteine, reduced. As an additional precaution, the cysteine and glutathione were resuspended and added fresh to the selection buffer just before use (early rounds) or the freshly prepared solution was frozen in single-use aliquots and thawed just before use (later rounds). Additionally, arginine was included in the cysteine/glutathione mix because arginine is also somewhat volatile in that its solutions absorb carbon dioxide from air, thus potentially changing the pH.

In vitro selection was performed for the purpose of discovering novel deoxyribozymes ideally suited for cleaving RNA in cells. Several new classes of magnesium dependent RNA-cleaving DNA sequences have been recovered. These enzymes show no primary sequence similarity to known deoxyribozymes performing the same chemical reaction. Prototypic unimolecular representatives from each of these classes self-cleave at imbedded RNA moieties with observed rate constants of ˜10⁻² min⁻¹ in 0.5 mM MgCl₂, with 150 mM KCl, 7 mM putrescine 10 mM reduced glutathione, and 50 mM HEPES pH 7.6 at 37° C. A shortened version of the sequence of the dominant class, class I, has a catalytic core of 20 nucleotides. This deoxyribozyme can be made to cleave the target in trans with a pseudo first order rate constant of at least 0.06 min⁻¹, can be generalized to cleave an in vitro transcribed RNA target of a different sequence, and has a pKₐ for a catalytically critical functional group of 7.9. Its kinetic rate increases in higher concentrations of magnesium but its maximum rate has yet to be determined.

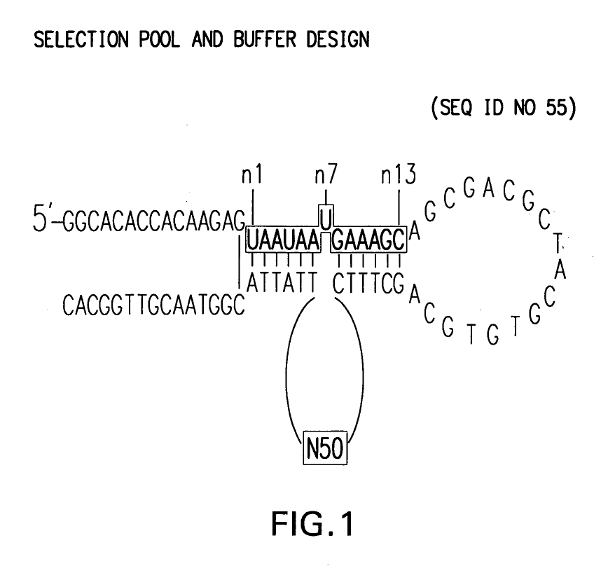

The construct on which the library was based is shown in FIG. 1 and the selection scheme utilized is shown in FIG. 4. Capitol letters indicate the four standard DNA bases and RNA bases (reverse type) in regions of the molecular library which were constant among all molecules. These regions provided the RNA substrate, positioned the random region adjacent to the substrate by base-pairing and allowed manipulation of the library during selection using standard methods. Catalytic sequences were derived from the region of 50 nucleotides, imbedded between the constant regions, synthesized to contain random DNA sequences.

Molecules were selected on the basis of their ability to self-cleave at an RNA residue. Molecules were reacted in solution in a buffer which approximated the composition of Escherichia coli cytoplasm with respect to conditions expected to be relevant to RNA cleavage (FIG. 1, legend). Incubations were performed at 37° C. Magnesium ions and amino acids were initially included in the selection buffer at concentrations well above cytoplasmic levels in order to favor RNA cleavage so as to increase the copy number of deoxyribozymes from the random library. Active catalysts were isolated from the remainder of the pool by size separation for all possible cleavage events within the RNA region with denaturing PAGE. Once a catalytic population had been established, the conditions of the selection were made increasingly more stringent by decreasing the time the molecules were allowed to react and by decreasing the concentration of all buffer components to physiologic levels.

No known DNAzyme motif has been identified amongst the six classes of DNAs examined from the current selection. Instead entirely new catalytic motifs have been discovered (Table 2) that have no detectable primary or secondary sequence similarity to known RNA-cleaving deoxyribozymes. Representative clones from each class were tested in their unimolecular format to determine catalytic rate and cleavage site (Table 3). These new sequences may represent unique tertiary structures having altered kinetic properties. Studies are in progress to assess each class individually.

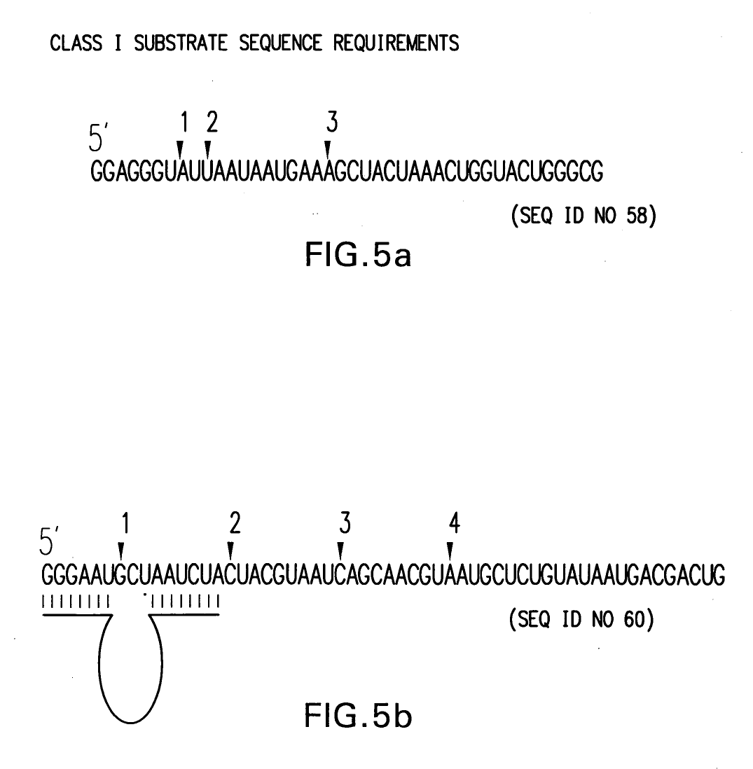

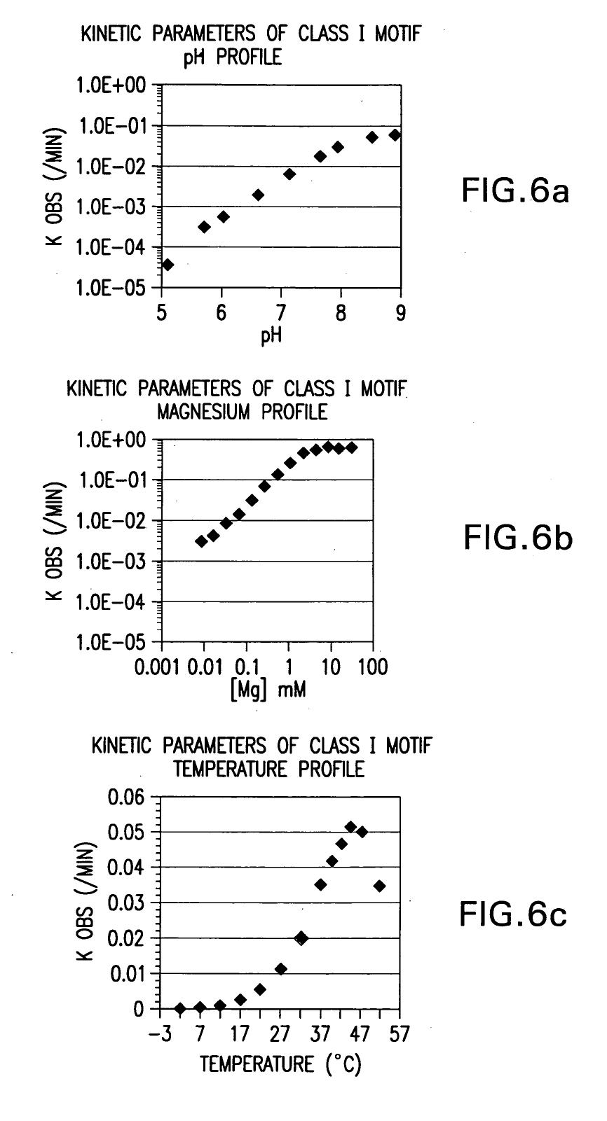

Clone 27 of class I was studied in a minimized, bimolecular format (FIG. 2). The secondary structure shown in FIG. 2a is based on the region of conserved nucleotides underlined in Table 2 with base-pairing restored at the 3′ end of the catalyst. The K_{d} for this enzyme interacting with substrate is 5 μM, which may reflect poor binding due to the A-U rich nature of the putative substrate binding arms. The enzyme does not require glutathione but does lose 10-fold activity in the absence of putrescine. Its pKₐ is ˜7.9, reaching a maximum pseudo first order rate constant in 0.5 mM Mg²⁺ of 0.06 min⁻¹. The enzyme also cleaves the in vitro transcribed target shown in FIG. 2b, demonstrating that it does not require DNA residues in the substrate and can be generalized by changing the substrate binding arms. The relative cleavage rates of the Class I motif on various substrates shown in FIG. 5 are presented in Table 4. Under single-turnover conditions, the rate of each enzyme is limited by binding a single magnesium ion with a Kd of 1 mM (FIG. 6). Therefore, the catalytic core is well adapted to the magnesium and pH levels in cells. The pseudo first order rate constant for RNA cleavage under simulated physiologic conditions (pH 7.6, 2 mM Mg²⁺, 150 mM K⁺, 7 mM putrescine, 37° C.) is 0.060 min⁻¹. The maximum rate constant at room temperature with Mg²⁺ increased to 8 mM and pH at 8.9 is 0.22 min⁻¹.

The Class I motif has been targeted to cleave a variety of RNA substrates under simulated physiologic, single-turnover conditions by altering the putative substrate binding regions to be complementary to new targets. Additional cleavage sites are shown in FIG. 5. The cleavage rates relative to the model substrate from FIG. 2 are summarized in Table 4 and show wide variation. This variation is not due to an inability to recognize an all-RNA substrate over an RNA/DNA chimera, as was present in the original selection but is most likely a result of intramolecular structure of the RNA targets. The substrate in FIG. 5A corresponds to a portion of the 5′ UTR and ORF of an mRNA of a bacterial protein while the substrate in FIG. 5B was specifically designed for this test. The suitability of the latter substrate was checked by the secondary structure prediction program mfold (Mathews et al., 1999, J. Mol. Biol., 288, 911-940) version 3.1 such that: (1) the substrate was predicted not to have significant intramolecular structure; (2) each deoxyribozyme would interact with only one site on the RNA; (3) and each deoxyribozyme/substrate base-pairing interaction would 4 to 8 kcal/mole more stable than the model substrate interaction indicated in FIG. 2. Nevertheless, tertiary and non-standard interactions that may have significant effects, especially intramolecularly, may not be predicted.

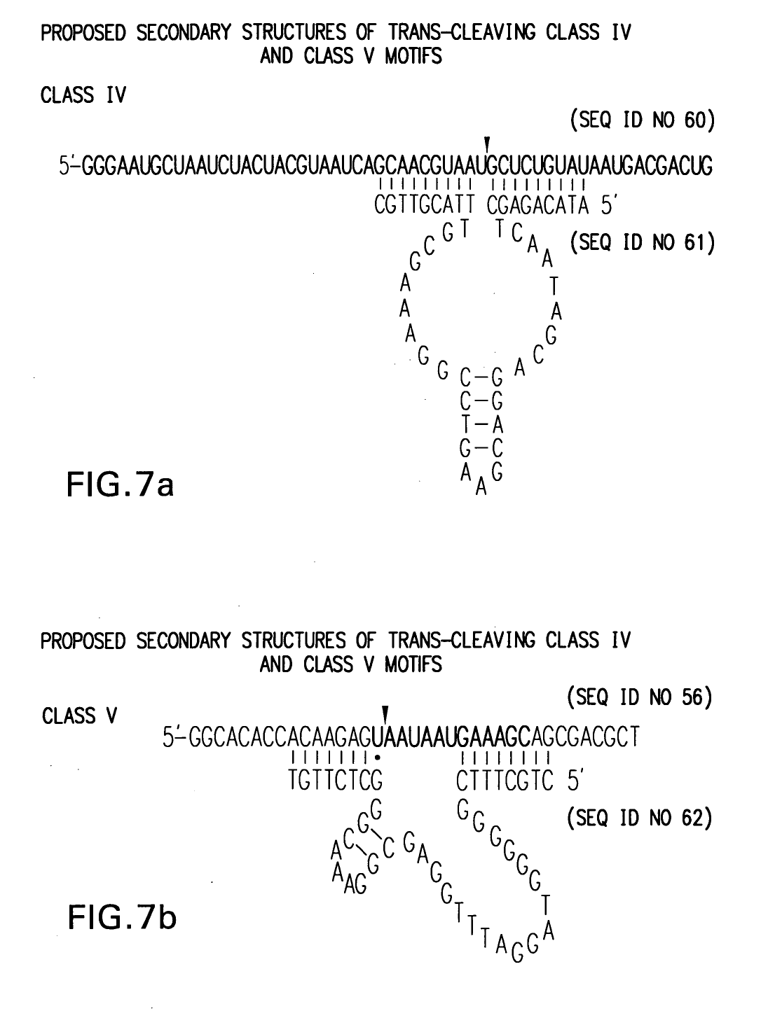

Class IV and class V motifs can also cleave substrates in trans (FIG. 7). The class IV clone sequences from in vitro selection contained an extended stem-loop in the core that has been truncated, making the core size no more than 29 nucleotides. Likewise, class V has been truncated to a core size of 28 nucleotides. The Class IV motif can be targeted to cleave an in vitro transcribed RNA with a pseudo first order rate constant of 0.002 min in 0.5 mM Mg2+, 150 mM K+, 7 mM putrescine, pH 7.6, 37° C. With this substrate, the Kd is approximately 1 μM in the same buffer. The motif has been shown to have no requirement for glutathione. The rate constant of the class V motif is estimated to be approximately 0.006 min−1 when tested in a similar buffer as above but with 10 mM glutathione included.

Studies were performed on clones 27 and 37 of class II. As for clone 27, an attempt was made to synthesize a molecule thought to be the minimal catalytically active sequence but with perfect substrate recognition sites (FIG. 3). There is a dramatic loss of activity with this construct. An unligated bimolecular assay on the original clone 37 where the entire sequence of the full-length molecule was present but the molecule was severed in the connecting loop region was also inactive.

The other motifs have been studied unimolecularly, cleaving RNA in cis. The class III clone, 21, does cleave in an unligated bimolecular assay. However, the poor procession of the unimolecular reaction (only 7% processed, Table 3) indicates an interference of some sort, possibly one that might be solved by truncation if more structural data were available. Clone 34 does show activity but apparently activates during elution from the purification gel following preparation. Clone 34 reactions show significant cleavage at time zero but no additional cleavage under several buffer conditions, including those designed to mimic the elution buffer. An isolated class VI motif has been shown to be a magnesium dependent catalyst with an estimated cleavage rate constant of 0.002 min−1 (0.5 mM Mg²⁺, 150 mM K⁺, 7 mM putrescine, 10 mM glutathione pH 7.6, 37° C.).

DNAzyme Engineering

Sequence, chemical and structural variants of DNAzymes of the present invention can be engineered using the techniques shown above and known in the art to cleave a separate target RNA or DNA in trans. DNAzymes can be reduced or increased in size using techniques known in the art. Techniques described for engineering molecules containing 2′-hydroxyl (2′-OH) groups can be applied to the DNA enzymes of the instant invention (for example, see Zaug et al., 1986. Nature, 324, 429; Ruffner et al., 1990, Biochem., 29, 10695; Beaudry et al., 1990, Biochem., 29, 6534; McCall et al. 1992, Proc. Natl. Acad. Sci., USA., 89, 5710; Long et al., 1994, supra; Hendry et al., 1994, BBA 1219, 405; Benseler et al., 1993, JACS, 115, 8483; Thompson et al., 1996, Nucl. Acids Res., 24, 4401; Michels et al., 1995, Biochem., 34, 2965; Been et al., 1992, Biochem., 31, 11843, Guo et al., 1995, EMBO. J., 14, 368; Pan et al., 1994, Biochem., 33, 9561; Cech, 1992, Curr. Op. Struc. Bio., 2, 605; Sugiyama et al., 1996, FEBS Lett., 392, 215; Beigelman et al., 1994, Bioorg. Med. Chem., 4, 1715; all of these references are incorporated in their totality by reference herein). For example, the stem-loop domains of the DNAzymes may not be essential for catalytic activity and hence can be systematically reduced in size using a variety of methods known in the art, to the extent that the overall catalytic activity of the DNAzyme is not significantly decreased. In addition, the introduction of variant stem-loop structures via site directed mutagenesis and/or chemical modification can be employed to develop DNAzymes with improved catalysis, increased stability, or both.

Further rounds of in vitro selection strategies described herein and variations thereof can be readily used by a person skilled in the art to evolve additional nucleic acid catalysts and such new catalysts are within the scope of the instant invention. Additionally, “Mutagenic PCR” (Cadwell R C, Joyce G F PCR Methods Appl 1994 Jun 3:6 S136-40) can be used to further optimize the sequences described in Formulae I and II. In addition, the optimization of these variant DNAzyme constructs by modification of stem-loop structures as is known in the art may provide for species with improved cleavage activity.

Target sequence requirements for DNAzymes can be determined and evaluated using methods known in the art.

Diagnostic Uses

Enzymatic nucleic acids of this invention can be used as diagnostic tools to examine genetic drift and mutations within diseased cells or to detect the presence of target RNA in a cell. The close relationship between DNAzyme activity and the structure of the target RNA allows the detection of mutations in any region of the molecule which alters the base-pairing and three-dimensional structure of the target RNA. By using multiple DNAzymes described in this invention, it is possible to map nucleotide changes which are important to RNA structure and function in vitro, as well as in cells and tissues. Cleavage of target RNAs with DNAzymes can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple DNAzymes targeted to different genes, DNAzymes coupled with known small molecule inhibitors, or intermittent treatment with combinations of DNAzymes and/or other chemical or biological molecules). Other in vitro uses of DNAzymes of this invention are well known in the art, and include detection of the presence of mRNAs associated with disease condition. Such RNA is detected by determining the presence of a cleavage product after treatment with a DNAzyme using standard methodology.

In a specific example, DNAzymes which cleave only wild-type or mutant forms of the target RNA are used for the assay. The first DNAzyme is used to identify wild-type RNA present in the sample and the second DNAzyme is used to identify mutant RNA in the sample. As reaction controls, synthetic substrates of both wild-type and mutant RNA are cleaved by both DNAzymes to demonstrate the relative DNAzyme efficiencies in the reactions and the absence of cleavage of the “non-targeted” RNA species. The cleavage products from the synthetic substrates also serve to generate size markers for the analysis of wild-type and mutant RNAs in the sample population. Thus each analysis can involve two DNAzymes, two substrates and one unknown sample which are combined into six reactions. The presence of cleavage products are determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype is adequate to establish risk. If probes of comparable specific activity are used for both transcripts, then a qualitative comparison of RNA levels will be adequate and will decrease the cost of the initial diagnosis. Higher mutant form to wild-type ratios will be correlated with higher risk whether RNA levels are compared qualitatively or quantitatively.

Additional Uses

Potential uses of sequence-specific enzymatic nucleic acid molecules of the instant invention include many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the DNAzyme is ideal for cleavage of RNAs of unknown sequence.

The nucleic acid catalysts of the instant invention can be used to specifically cleave an RNA sequence for which an appropriately engineered nucleic acid catalyst base pairs at the designated flanking regions (e.g., X and Y in Formulae I and II). Suitable target RNA substrates include viral, messenger, transfer, ribosomal, nuclear, organellar, other cellular RNA, or any other natural RNA having a cleavage sequence, as well as RNAs, which have been engineered to contain an appropriate cleavage sequence. The nucleic acid catalysts are useful in vivo in prokaryotes or eukaryotes of plant or animal origin for controlling viral infections or for regulating the expression of specific genes.

Once introduced into the cell, the nucleic acid catalyst binds to and cleaves the target RNA sequence or sequences for which it has been designed, inactivating the RNA. If the RNA is necessary for the life cycle of a virus, the virus will be eliminated and if the RNA is the product of a specific gene, the expression of that gene will be regulated. The nucleic acid catalyst can be designed to work in prokaryotes and within the nucleus (without poly(A) tail) or in the cytoplasm of a eukaryotic cell (with polyadenylation signals in place) for plants and animals.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Thus, additional embodiments are within the scope of the invention and within the claims that follow:

TABLE 1

| | | | | | |
|---|---|---|---|---|---|
| | | | | Wait Time* 2′- | Wait Time* |
| Reagent | Equivalents | Amount | Wait Time* DNA | O-methyl | RNA |

A. 2.5 μmol Synthesis Cycle ABI 394 Instrument

| | | | | | |
|---|---|---|---|---|---|
| Phosphoramidites | 6.5 | 163 μL | 45 sec | 2.5 min | 7.5 min |
| S-Ethyl Tetrazole | 23.8 | 238 μL | 45 sec | 2.5 min | 7.5 min |
| Acetic Anhydride | 100 | 233 μL | 5 sec | 5 sec | 5 sec |
| N-Methyl Imidazole | 186 | 233 μL | 5 sec | 5 sec | 5 sec |
| TCA | 176 | 2.3 mL | 21 sec | 21 sec | 21 sec |
| Iodine | 11.2 | 1.7 mL | 45 sec | 45 sec | 45 sec |
| Beaucage | 12.9 | 645 μL | 100 sec | 300 sec | 300 sec |
| Acetonitrile | NA | 6.67 mL | NA | NA | NA |

B. 0.2 μmol Synthesis Cycle ABI 394 Instrument

| | | | | | |
|---|---|---|---|---|---|
| Phosphoramidites | 15 | 31 μL | 45 sec | 233 sec | 465 sec |
| S-Ethyl Tetrazole | 38.7 | 31 μL | 45 sec | 233 min | 465 sec |
| Acetic Anhydride | 655 | 124 μL | 5 sec | 5 sec | 5 sec |
| N-Methyl Imidazole | 1245 | 124 μL | 5 sec | 5 sec | 5 sec |
| TCA | 700 | 732 μL | 10 sec | 10 sec | 10 sec |
| Iodine | 20.6 | 244 μL | 15 sec | 15 sec | 15 sec |
| Beaucage | 7.7 | 232 μL | 100 sec | 300 sec | 300 sec |
| Acetonitrile | NA | 2.64 mL | NA | NA | NA |

C. 0.2 μmol Synthesis Cycle 96 well Instrument

| | Equivalents | Amount | Wait Time* | Wait Time* 2′-O- | |
|---|---|---|---|---|---|
| Reagent | DNA/2′-O-methyl/Ribo | DNA/2′-O-methyl/Ribo | DNA | methyl | Wait Time* Ribo |
| Phosphoramidites | 22/33/66 | 40/60/120 μL | 60 sec | 180 sec | 360 sec |
| S-Ethyl Tetrazole | 70/105/210 | 40/60/120 μL | 60 sec | 180 min | 360 sec |
| Acetic Anhydride | 265/265/265 | 50/50/50 μL | 10 sec | 10 sec | 10 sec |
| N-Methyl Imidazole | 502/502/502 | 50/50/50 μL | 10 sec | 10 sec | 10 sec |
| TCA | 238/475/475 | 250/500/500 μL | 15 sec | 15 sec | 15 sec |
| Iodine | 6.8/6.8/6.8 | 80/80/80 μL | 30 sec | 30 sec | 30 sec |
| Beaucage | 34/51/51 | 80/120/120 | 100 sec | 200 sec | 200 sec |
| Acetonitrile | NA | 1150/1150/1150 μL | NA | NA | NA |

*Wait time does not include contact time during delivery.

TABLE 2

SEQUENCES OF ACTIVE DEOXYRIBOZYME CLASSES

| | | | | |
|---|---|---|---|---|
| Sequence | | | Activity of | SEQ |
| Class | Clone | Sequence | clone | ID NO |

| Class I | G14-p3 | GATGCAGCTGGGGAGGCGTTTACTTAGATGTGTGACGTAACCGTGTAGG | | 1 |
|---|---|---|---|---|
| | G14-p10 | GATGCAGCTGAGGAGGCGTTTCTGTTGGCGGTGTCTCGAGTTAGAACCTC | | 2 |
| | G14-p15 | GATGCAGCTGAGGAGGCGTTTCTGTTGGCGGTGTCTCGAGTTAGAATCT | | 3 |
| | G14-p19 | GATGCAGCTGAGGAGGCGTTTCTGTTGGCGGTGTCTTGAGTTAGAACCTC | | 4 |
| | G14-p24 | GATGCAGCTGAGGAGGCGTTTCTGTTGGTGGTGTCTTGAGTAGAACCTC | | 5 |
| | G21-p23 | GATGCAGCTGGGGAGGCGTTTACTTAGATGTGTGATTTCATGTTGG | Active | 6 |
| | G21-p25 | GATGCAGCTGGGGAGGCGTTTACTCGGGAAAATGTGATGTGTCCTT | | 7 |
| | G21-p26 | GATGCAGCTGGGGAGGCGTTTACTCTGGAAAGTGTGATCTGTTCTG | | 8 |
| | G21-p27,p79 | GATGCAGCTGGGGAGGCGTTTACTCGGGAAAATGTGATGTGTTCTG | 0.024 min⁻¹ | 9 |
| | G21-p29 | GATGCAGCTGGGGAGGCGTTTACTCGGGAAAATGTGATGCCTTTTG | | 10 |
| | G21-p30 | GATGCAGCTGGGGAGGCGTTTACTCGGGAAAATGTGATGCCTCTCG | | 11 |
| | G21-p40 | GATGCAGCTGGGGAGGCGTTTACTTAGATGTGTGACGTATCTCTTC | | 12 |
| | G21-p41,p78, | GATGCAGCTGGGGAGGCGTTTACTCGGGAAAATGTGATGCCTCTCG | | 13 |
| | p83 | | | |
| | G21-p44 | GATGCAGCTGGGGAGGCGTTTACTCGGGAAAATGTGATGTGTTCTA | | 14 |
| | G21-p45 | GATGCAGCTGGGGAGGCGTTTACTCTGGAAAATGTGATGTCTCTTG | | 15 |
| | G21-p48 | GATGCAGCTGGGGAGGCGTTTACTCGGGAAAATGTGATGTGTCTTG | | 16 |
| | G21-p51 | GATGCAGCTGAGGAGGCGTTTCTGTTGGTGGTGTCTCGAGTTATTA | Active | 17 |
| | G21-p57 | GATGCAGCTGGGGAGGCGTTTACTTAGATGTGTGTTGCCTGTATT | | 18 |
| | G21-p58 | GATGCAGCTGGGGAGGCGTTTACTTAGATGTGTGACGTAACTGCGTAGG | | 19 |
| | G21-p60 | GATGCAGCTGGGGAGGCGTTTACTTAGATGTGTGTCATAACTCAC | | 20 |
| | G21-p64,p86 | GATGCAGCTGGGGAGGCGTTTACTCTGGAAAATGTGATGTGTTCTG | | 21 |
| | G21-p68 | GATGCAGCTGGGGAGGCGTTTACTCTGGAAAATGTGATGTGTTCTAT | | 22 |
| | G21-p71 | GATGCAGCTGAGGAGGCGTTTATCTTGTGTTTATTGGATTTGGCTC | | 23 |
| | G21-p72 | GATGCAGCTGGGGAGGCGTTTACTCGGGAAAATGTGATGTGTTTTG | | 24 |
| | G21-p81 | GATGCAGCTGGGGAGGCGTTTACTCGGGAAATGTGATGTGTTCTG | | 25 |
| | G21-p82 | GATGCAGCTGAGGAGGCGTTTCTGTTAAAATTGTATATTGTATCTT | | 26 |
| | G21-p88 | GATGCAGCTGGGGAGGCGTTTACTCTGGAAAATGTGTTGTGTTCCT | | 27 |
| Class II | G21-p37,p62 | GGGCAACCTGAGGGTGGGGAAGCACAGGGTATTTCTCCGTAGTGGTCGG | 0.0074 min⁻¹ | 28 |
| | G21-p42 | GGGGAACCTGAGGGTGGGGAAGCTCTGGGTATTTCTCGGTAGTGGTCGG | | 29 |
| | G21-p47 | GGGGAGCGTTAGTGGGGAAGCACAGGGTTGTACCTATTGTGGGTTGCCT | Active | 30 |
| | G21-p53,p61 | GGGGAAGGAAGCATCTTATGCGACCTGGGGAAGCACATGGTTAATTCTC | Active | 31 |
| Class III | G21-p21 | GGGGGGGGTTTTACTAGGGGGGGAATAGGGAGTTCCCCCACTGGTCTGG | 0.0014 min⁻¹ | 32 |
| Class IV | G21-p32,p43 | TCAATAGCAGGACGACTGTATATAAAGTGTGTGGTTCGTCCGGAAAGCGT | 0.0093 min⁻¹ | 33 |
| | G21-p50 | TCAATAGCAGGACGACTGCATATAAAGTCTGTGGTTTGTCCGGAAAGCGT | | 34 |
| | G21-p63,p65 | TCAATACCAGGACGACCGCATATAAAGTGTGTGGCTTGTCCGCAAAGCGT | | 35 |
| | G21-p69 | TCAATAGCAGGACGATTGCATATAAAGTGTGTGGCTCGTCCGGAAAGCGT | | 36 |
| | G21-p77 | TCAATAGCAGGACGACCGCATATAAAGTGTGTGGTTTGTCCGGAAAGCGT | | 37 |
| | G21-p85 | TCAATAGCAGGACGACTGTATACAAAGTGTGTGGTTTGTCCGGAAAGCGT | | 38 |
| Class V | G14-p18 | -GGGGGTAGGATTTGGAGCGGAGACGGGCTCTTGGGGTGTTGTATGCCCA | | 39 |
| | G21-p52 | GGGGGGTAGGATTTGGAGCGGAAACGGGCTCTTGGGGGTCTGCT | 0.0014 min⁻¹ | 40 |
| | G21-p55 | GGGGGGTAGGATTTGGAGCGGAAACGGGCTCTTGGGGAGTCTTG | | 41 |
| | G21 -p74 | GGGGGGTACGATTTGGAGCGGAAACGGGCTCTTGGGGGTCTGCC | | 42 |
| Class XI | G14-p21 | ACAGGTGGTGTCGGCGCATTCATAGAGGGTTGCTTGGTGGTTATGCACA | 0.002 min⁻¹ | 43 |
| Sequences are displayed 5′ to 3′. Likely catalytic core regions are underlined based on truncated bimolecular constructs or on conservation with other molecules in the class. | | | | |
| Reaction conditions unimolecular constructs, 0.5 mM MgCL₂, 150 mM KCl, 7 mM putrescine, 10 mM glutathione, 50 mM HEPES pH 7.6, 37° C. | | | | |

TABLE 3
Kinetic unimolecular parameters of selected clones

| | | | Observed | |
|---|---|---|---|---|
| | | Cleavage | rate | Amount |
| Class | Clone | site | constant | Processed |
| I | 23 | nt 4 | nd | Nd |
| | 27 | nt 4 | 0.024 | 80% |
| | 51 | nt 4 | 0.0048 | nd |
| II | 37 | nt 6 | 0.0074 | 70% |
| | 47 | nt 6 | nd | Nd |
| | 53 | nt 6 | nd | Nd |
| III | 21 | nt 4 | 0.014 | 7% |
| IV | 32 | nt 7 | 0.093 | 50% |
| V | 52 | nt 1 | 0.014 | 70% |
| VI | 33 | Inactive | nd | nd |
| | 34 | nt 7^{§} | nd | nd |

Reaction conditions: 0.5 mM MgCl₂, 7 mM putrescine, 10 mM glutathione, 50 mM HEPES pH 7.6, 37° C.
Cleavage site: enzyme cleaves after the indicated nucleotide, numbered 1 to 13 (FIG. 1) within the RNA region.
Observed rate constant is in units of min⁻¹.
Amount processed is the amount of cleavage at which the reaction plateaus.
*Reaction kinetics changed during the time course such the new reaction did not asymptotically approach a minimum.
^{&Ovalhollow;}Enzyme had reacted prior to assay and did not react further. Presumably it reacted during elution from the purification gel but these conditions could not be duplicated.

TABLE 4

RELATIVE CLEAVAGE RATES OF MOTIF MDR1 ON VARIOUS SUBSTRATES

| | | | |
|---|---|---|---|
| Substrate name | Substrate squence | Relative rate of cleavage* | SEQ ID NO |

| | | | |
|---|---|---|---|
| Model, FIG. 2 | aagagUAA-UAA-UGAAAGCag | 1 | 44 |
| FIG. 5a, site 1 | GAGGGUAU-UAA-UAAUGAAAG | 0.18 | 45 |
| FIG. 5a, site 2 | GGUAUUAA-UAA-UGAAAGCUA | 0.30 | 46 |
| FIG. 5b, site 1 | GGGAAUGC-UAA-UCUACUAGG | 0.0057 | 47 |
| FIG. 5b, site 2 | CUACUACG-UAA-UCAGCAACG | 0 | 48 |
| FIG. 5b, site 3 | CAGCAACG-UAA-UGCUCUGUA | 0.011 | 49 |
| FIG. 5b, site 4 | GCUGUGUA-UAA-UGACGACUG | 0 | 50 |

Legend:
The catalytic core from G1 to T19 shown in FIG. 2 was synthesized between arms that base-pair to the substrate sequence such that the U immediately 3′ to the cleavage site is engaged in a wobble pair with G1 of the catalytic core and the UAA separately by hyphens is bulged. Cleavage site is 3′ to the U of the bulged UAA. Substrates are RNA except for the model substrate where DNA is indicated by lowercase.
Assay conditions:
50 μM enzyme, trace (< 5) mM substrate, 2 mM Mg²⁻, 150 mM K⁺, 7 mM putrescine, pH 7.6, 37° C.
*This data is meant for comparison only. Assays for the substrate sites in FIG. 5B are not multiple-point rate assays and the rate constant is estimated from a single time point. For this reason, the actual rate constants or estimated rate constants have been normalized to the rate for the model substrate so that the actual values will not be emphasized.

What is claimed is:

1. A nucleic acid molecule with endonuclease activity having the formula I:
3′-X-Z-Y-5′
wherein, X and Y are independently oligonucleotides of lengths sufficient to stably interact with a target nucleic acid molecule, and Z is independently a nucleotide sequence comprising 5′-GATGCAGCTGGGGAGGCGTTT-3′ (SEQ ID NO 51).

2. A nucleic acid molecule with endonuclease activity having the formula II:
3′-X-W-V-R-Y-5′
wherein, X and Y are independently oligonucleotides of length sufficient to stably interact with a target nucleic acid molecule, R is independently a nucleotide sequence comprising 5′-GGGGA-3′, V represents a nucleotide or non-nucleotide linker, which may be present or absent; W is independently an oligonucleotide comprising a nucleotide sequence selected from the group consisting of 5′-TGGGGAAGCACAGGGT-3′ (SEQ ID NO 52), 5′-TGGGGAAGCTCTGGGT-3′ (SEQ ID NO 53), 5′-TGGGGAAGCACAGGGT-3′ (SEQ ID NO 54), and 5′-TGGGGAAGCACAGGGT-3′(SEQ ID NO 55).

3. The nucleic acid molecule of claim 2, wherein V is a nucleotide linker.

4. The nucleic acid molecule of claim 2, wherein V is a non-nucleotide linker.

5. The nucleic acid molecule of claim 3, wherein said nucleotide linker is a sequence selected from the group consisting of 5′-ACCTGAGGG-3′, 5′-GCGTTAG-3′ and 5′-AGGAAGCATCTTATGCGACC-3′(SEQ ID NO 56).

6. The nucleic acid molecule of claim 3, wherein said nucleotide linker is a nucleic acid aptamer.

7. The nucleic acid molecule of claim 6, wherein said aptamer is an ATP aptamer.

8. The nucleic acid molecule of claim 1 or claim 2, wherein said chemical linkage is independently or in combination selected from the group consisting of phosphate ester amide, phosphorothioate, phosphorodithioate, methylphosphonate, arabino, and arabinofluoro linkages.

9. The nucleic acid molecule of claim 1 or claim 2, wherein said nucleic acid molecule is chemically synthesized.

10. The nucleic acid molecule of claim 1 or claim 2, wherein said nucleic acid molecule comprises at least one sugar modification.

11. The nucleic acid molecule of claim 1 or claim 2, wherein said nucleic acid molecule comprises at least one nucleic acid base modification.

12. The nucleic acid molecule of claim 1 or claim 2, wherein said nucleic acid molecule comprises at least one phosphate backbone modification.

13. The nucleic acid molecule of claim 10, wherein said sugar modification is a 2′-modification.

14. The nucleic acid molecule of claim 13, wherein said sugar modification is selected from the group consisting of 2′-O-methyl, 2′-O-allyl, 2′-C-allyl, 2′-deoxy-2′-fluoro, 2′-deoxy-2′-arabinofluoro, 2′-deoxy-2′-amino, and 2′-O-amino modifications.

15. The nucleic acid molecule of claim 12, wherein said phosphate backbone modification is selected from the group consisting of phosphorothioate, phosphorodithioate, methylphosphonate, and amide modifications.

16. The nucleic acid molecule of claim 1 or claim 2, wherein said nucleic acid molecule comprises a 5′-cap, a 3′-cap, or both a 5′-cap and a 3′-cap.

17. The nucleic acid molecule of claim 16, wherein said 5′-cap is a phosphorothioate modification of at least one 5′-terminal nucleotide in said nucleic acid molecule.

18. The nucleic acid molecule of claim 16, wherein said 5′-cap is a phosphorothioate modification of at least two 5′-terminal nucleotides in said nucleic acid molecule.

19. The nucleic acid molecule of claim 16, wherein said 5′-cap is a phosphorothioate modification of at least three 5′-terminal nucleotides in said nucleic acid molecule.

20. The nucleic acid molecule of claim 16, wherein said 5′-cap is a phosphorothioate modification of at least four 5′-terminal nucleotides in said nucleic acid molecule.

21. The nucleic acid molecule of claim 16, wherein said 3′-cap is a 3′-3′ inverted riboabasic moiety.

22. The nucleic acid molecule of claim 16, wherein said 3′-cap is a 3′-3′ inverted deoxyriboabasic moiety.

23. The nucleic acid molecule of claim 1 or claim 2, wherein said nucleic acid cleaves a separate nucleic acid molecule.

24. The nucleic acid molecule of claim 23, wherein said separate nucleic acid molecule is RNA.

25. The nucleic acid molecule of claim 23, wherein said nucleic acid comprises between 12 and 100 bases complementary to said separate nucleic acid molecule.

26. The nucleic acid molecule of claim 23, wherein said nucleic acid comprises between 14 and 24 bases complementary to said separate nucleic acid molecule.

27. The nucleic acid molecule of claim 1 or claim 2, wherein X and Y are independently lengths selected from the group consisting of 3, 4, 5, 6, 7, 8, 9, 10, 11, 12, 13, 14, 15, 17, and 20 nucleotides.

28. The nucleic acid molecule of claim 1 or claim 2, wherein the length of X is equal to the length of Y.

29. The nucleic acid molecule of any of claims 1 and 2, wherein the length of X is not equal to the length of Y.

30. A cell including the nucleic acid molecule of claim 1 or claim 2.

31. The cell of claim 30, wherein said cell is a prokaryotic cell.

32. The cell of claim 30, wherein said cell is a eukaryotic cell.

33. The cell of claim 30, wherein said cell is a mammalian cell.

34. The cell of claim 33, wherein said cell is a human cell.

35. The cell of claim 30, wherein said cell is a plant cell.

36. An expression vector comprising a nucleic acid sequence encoding at least one of the nucleic acid molecules of claim 1 or claim 2, in a manner which allows expression of that nucleic acid molecule.

37. A cell including the expression vector of claim 36.

38. The cell of claim 37, wherein said cell is a mammalian cell.

39. The cell of claim 38, wherein said cell is a human cell.

40. A pharmaceutical composition comprising the nucleic acid molecule of claim 1 or claim 2.

41. A method for modulating expression of a gene in a plant cell by administering to said cell the nucleic acid molecule of claim 1 or claim 2.

42. A method for modulating expression of gene in a mammalian cell by administering to said cell the nucleic acid molecule of claim 1 or claim 2.

43. A method for modulating expression of a gene in a bacterial cell by administering to said cell the nucleic acid molecule of claim 1 or claim 2.

44. A method for modulating expression of a gene in a fungal cell by administering to said cell the nucleic acid molecule of claim 1 or claim 2.

45. A method of cleaving a target nucleic acid comprising, contacting the nucleic acid molecule of claim 1 or claim 2 with said target nucleic acid under conditions suitable for the cleavage of said target nucleic acid.

46. The method of claim 45, wherein said cleavage is carried out in the presence of a divalent cation.

47. The method of claim 46, wherein said divalent cation is Mg²⁺.


SEQUENCE LISTING (67 sequences)

SEQ ID NO: 1 - DNA, 49 bp, Artificial Sequence
  Description of Artificial Sequence Deoxyribozyme
gatgcagctggggaggcgtttacttagatgtgtgacgtaaccgtgtagg

SEQ ID NO: 2 - DNA, 50 bp, Artificial Sequence
  Description of Artificial Sequence Deoxyribozyme
gatgcagctgaggaggcgtttctgttggcggtgtctcgagttagaacctc

SEQ ID NO: 3 - DNA, 49 bp, Artificial Sequence
  Description of Artificial Sequence Deoxyribozyme
gatgcagctgaggaggcgtttctgttggcggtgtctcgagttagaatct

SEQ ID NO: 4 - DNA, 50 bp, Artificial Sequence
  Description of Artificial Sequence Deoxyribozyme
gatgcagctgaggaggcgtttctgttggcggtgtcttgagttagaacctc

SEQ ID NO: 5 - DNA, 49 bp, Artificial Sequence
  Description of Artificial Sequence Deoxyribozyme
gatgcagctgaggaggcgtttctgttggtggtgtcttgagtagaacctc

SEQ ID NO: 6 - DNA, 46 bp, Artificial Sequence
  Description of Artificial Sequence Deoxyribozyme
gatgcagctggggaggcgtttacttagatgtgtgatttcatgttgg

SEQ ID NO: 7 - DNA, 46 bp, Artificial Sequence
  Description of Artificial Sequence Deoxyribozyme
gatgcagctggggaggcgtttactcgggaaaatgtgatgtgtcctt

SEQ ID NO: 8 - DNA, 46 bp, Artificial Sequence
  Description of Artificial Sequence Deoxyribozyme
gatgcagctggggaggcgtttactctggaaagtgtgatctgttctg

SEQ ID NO: 9 - DNA, 46 bp, Artificial Sequence
  Description of Artificial Sequence Deoxyribozyme
gatgcagctggggaggcgtttactcgggaaaatgtgatgtgttctg

SEQ ID NO: 10 - DNA, 46 bp, Artificial Sequence
  Description of Artificial Sequence Deoxyribozyme
gatgcagctggggaggcgtttactcgggaaaatgtgatgccttttg

SEQ ID NO: 11 - DNA, 46 bp, Artificial Sequence
  Description of Artificial Sequence Deoxyribozyme
gatgcagctggggaggcgtttactcgggaaaatgtgatgcctctcg

SEQ ID NO: 12 - DNA, 46 bp, Artificial Sequence
  Description of Artificial Sequence Deoxyribozyme
gatgcagctggggaggcgtttacttagatgtgtgacgtatctcttc

SEQ ID NO: 13 - DNA, 46 bp, Artificial Sequence
  Description of Artificial Sequence Deoxyribozyme
gatgcagctggggaggcgtttactcgggaaaatgtgatgtgttcta

SEQ ID NO: 14 - DNA, 46 bp, Artificial Sequence
  Description of Artificial Sequence Deoxyribozyme
gatgcagctggggaggcgtttactctggaaaatgtgatgtctcttg

SEQ ID NO: 15 - DNA, 46 bp, Artificial Sequence
  Description of Artificial Sequence Deoxyribozyme
gatgcagctggggaggcgtttactcgggaaaatgtgatgtgtcttg

SEQ ID NO: 16 - DNA, 46 bp, Artificial Sequence
  Description of Artificial Sequence Deoxyribozyme
gatgcagctgaggaggcgtttctgttggtggtgtctcgagttatta

SEQ ID NO: 17 - DNA, 45 bp, Artificial Sequence
  Description of Artificial Sequence Deoxyribozyme
gatgcagctggggaggcgtttacttagatgtgtgttgcctgtatt

SEQ ID NO: 18 - DNA, 49 bp, Artificial Sequence
  Description of Artificial Sequence Deoxyribozyme
gatgcagctggggaggcgtttacttagatgtgtgacgtaactgcgtagg

SEQ ID NO: 19 - DNA, 45 bp, Artificial Sequence
  Description of Artificial Sequence Deoxyribozyme
gatgcagctggggaggcgtttacttagatgtgtgtcataactcac

SEQ ID NO: 20 - DNA, 46 bp, Artificial Sequence
  Description of Artificial Sequence Deoxyribozyme
gatgcagctggggaggcgtttactctggaaaatgtgatgtgttctg

SEQ ID NO: 21 - DNA, 47 bp, Artificial Sequence
  Description of Artificial Sequence Deoxyribozyme
gatgcagctggggaggcgtttactctggaaaatgtgatgtgttctat

SEQ ID NO: 22 - DNA, 46 bp, Artificial Sequence
  Description of Artificial Sequence Deoxyribozyme
gatgcagctgaggaggcgtttatcttgtgtttattggatttggctc

SEQ ID NO: 23 - DNA, 46 bp, Artificial Sequence
  Description of Artificial Sequence Deoxyribozyme
gatgcagctggggaggcgtttactcgggaaaatgtgatgtgttttg

SEQ ID NO: 24 - DNA, 45 bp, Artificial Sequence
  Description of Artificial Sequence Deoxyribozyme
gatgcagctggggaggcgtttactcgggaaatgtgatgtgttctg

SEQ ID NO: 25 - DNA, 46 bp, Artificial Sequence
  Description of Artificial Sequence Deoxyribozyme
gatgcagctgaggaggcgtttctgttaaaattgtatattgtatctt

SEQ ID NO: 26 - DNA, 46 bp, Artificial Sequence
  Description of Artificial Sequence Deoxyribozyme
gatgcagctggggaggcgtttactctggaaaatgtgttgtgttcct

SEQ ID NO: 27 - DNA, 49 bp, Artificial Sequence
  Description of Artificial Sequence Deoxyribozyme
ggggaacctgagggtggggaagcacagggtatttctcggtagtggtcgg

SEQ ID NO: 28 - DNA, 49 bp, Artificial Sequence
  Description of Artificial Sequence Deoxyribozyme
ggggaacctgagggtggggaagctctgggtatttctcggtagtggtcgg

SEQ ID NO: 29 - DNA, 49 bp, Artificial Sequence
  Description of Artificial Sequence Deoxyribozyme
ggggagcgttagtggggaagcacagggttgtagctattgtgggttgcct

SEQ ID NO: 30 - DNA, 49 bp, Artificial Sequence
  Description of Artificial Sequence Deoxyribozyme
ggggaaggaagcatcttatgcgacctggggaagcacatggttaattctc

SEQ ID NO: 31 - DNA, 49 bp, Artificial Sequence
  Description of Artificial Sequence Deoxyribozyme
ggggggggttttactagggggggaatagggagttcccccactggtctgg

SEQ ID NO: 32 - DNA, 50 bp, Artificial Sequence
  Description of Artificial Sequence Deoxyribozyme
tcaatagcaggacgactgtatataaagtgtgtggttcgtccggaaagcgt

SEQ ID NO: 33 - DNA, 50 bp, Artificial Sequence
  Description of Artificial Sequence Deoxyribozyme
tcaatagcaggacgactgcatataaagtgtgtggtttgtccggaaagcgt

SEQ ID NO: 34 - DNA, 50 bp, Artificial Sequence
  Description of Artificial Sequence Deoxyribozyme
tcaatagcaggacgaccgcatataaagtgtgtggcttgtccggaaagcgt

SEQ ID NO: 35 - DNA, 50 bp, Artificial Sequence
  Description of Artificial Sequence Deoxyribozyme
tcaatagcaggacgattgcatataaagtgtgtggctcgtccggaaagcgt

SEQ ID NO: 36 - DNA, 50 bp, Artificial Sequence
  Description of Artificial Sequence Deoxyribozyme
tcaatagcaggacgaccgcatataaagtgtgtggtttgtccggaaagcgt

SEQ ID NO: 37 - DNA, 50 bp, Artificial Sequence
  Description of Artificial Sequence Deoxyribozyme
tcaatagcaggacgactgtatacaaagtgtgtggtttgtccggaaagcgt

SEQ ID NO: 38 - DNA, 49 bp, Artificial Sequence
  Description of Artificial Sequence Deoxyribozyme
gggggtaggatttggagcggagacgggctcttggggtgttgtatgccca

SEQ ID NO: 39 - DNA, 44 bp, Artificial Sequence
  Description of Artificial Sequence Deoxyribozyme
ggggggtaggatttggagcggaaacgggctcttgggggtctgct

SEQ ID NO: 40 - DNA, 44 bp, Artificial Sequence
  Description of Artificial Sequence Deoxyribozyme
ggggggtaggatttggagcggaaacgggctcttggggagtcttg

SEQ ID NO: 41 - DNA, 44 bp, Artificial Sequence
  Description of Artificial Sequence Deoxyribozyme
ggggggtaggatttggagcggaaacgggctcttgggggtctgcc

SEQ ID NO: 42 - DNA, 49 bp, Artificial Sequence
  Description of Artificial Sequence Deoxyribozyme
acaggtggtgtcggcgcattcatagagggttgcttggtggttatgcaca

SEQ ID NO: 43 - DNA, 20 bp, Artificial Sequence
  Description of Artificial Sequence Substrates for motif MDR1
aagaguaauaaugaaagcag

SEQ ID NO: 44 - RNA, 20 nt, Artificial Sequence
  Description of Artificial Sequence Substrates for motif MDR1
gaggguauuaauaaugaaag

SEQ ID NO: 45 - RNA, 20 nt, Artificial Sequence
  Description of Artificial Sequence Substrates for motif MDR1
gguauuaauaaugaaagcua

SEQ ID NO: 46 - RNA, 20 nt, Artificial Sequence
  Description of Artificial Sequence Substrates for motif MDR1
gggaaugcuaaucuacuacg

SEQ ID NO: 47 - RNA, 20 nt, Artificial Sequence
  Description of Artificial Sequence Substrates for motif MDR1
cuacuacguaaucagcaacg

SEQ ID NO: 48 - RNA, 20 nt, Artificial Sequence
  Description of Artificial Sequence Substrates for motif MDR1
cagcaacguaaugcucugua

SEQ ID NO: 49 - RNA, 20 nt, Artificial Sequence
  Description of Artificial Sequence Substrates for motif MDR1
gcucuguauaaugacgacug

SEQ ID NO: 50 - DNA, 21 bp, Artificial Sequence
  Description of Artificial Sequenc Possible catalytic core
gatgcagctggggaggcgttt

SEQ ID NO: 51 - DNA, 16 bp, Artificial Sequence
  Description of Artificial Sequence Possible catalytic core
tggggaagcacagggt

SEQ ID NO: 52 - DNA, 16 bp, Artificial Sequence
  Description of Artificial Sequence Possible catalytic core
tggggaagctctgggt

SEQ ID NO: 53 - DNA, 16 bp, Artificial Sequence
  Description of Artificial Sequence Possible catalytic core
tggggaagcacatggt

SEQ ID NO: 54 - DNA, 20 bp, Artificial Sequence
  Description of Artificial Sequence Nucleotide linker sequence
aggaagcatcttatgcgacc

SEQ ID NO: 55 - DNA, 123 bp, Artificial Sequence
  Description of Artificial Sequence  Chimeric selection pool molecule
ggcacaccacaagaguaauaaugaaagcagcgacgctacgtgtgcagctttcnnnnnnnnnnnnnnnnnnnnnnnnnnnnnnnnnnnnnnnnnnnnnnnnnnttattacggtaacgttggcac

SEQ ID NO: 56 - DNA, 37 bp, Artificial Sequence
  Description of Artificial Sequence Synthetic RNA/DNA chimera
ggcacaccacaagaguaauaaugaaagcagcgacgct

SEQ ID NO: 57 - DNA, 35 bp, Artificial Sequence
  Description of Artificial Sequence  Class I Enzymatic Nucleic Acid
ctgctttcgatgcagctggggaggcgtttactctt

SEQ ID NO: 58 - RNA, 42 nt, Artificial Sequence
  Description of Artificial Sequence  5′ UTR and ORF of E. colilipoprotein
ggaggguauuaauaaugaaagcuacuaaacugguacugggcg

SEQ ID NO: 59 - DNA, 45 bp, Artificial Sequence
  Description of Artificial Sequence  Class II Enzymatic Nucleic Acid
ctgctttcggggaacctgagggtggggaagcacagggttattact

SEQ ID NO: 60 - RNA, 56 nt, Artificial Sequence
  Description of Artificial Sequence  Class I substrate molecule
gggaaugcuaaucuacuacguaaucagcaacguaaugcucuguauaaugacgacug

SEQ ID NO: 61 - DNA, 47 bp, Artificial Sequence
  Description of Artificial Sequence Enzymatic Nucleic Acid
atacagagctcaatagcaggacgaagtccggaaagcgtttacgttgc

SEQ ID NO: 62 - DNA, 43 bp, Artificial Sequence
  Description of Artificial Sequence Enzymatic Nucleic Acid
ctgctttcggggggtaggatttggagcggaaacgggctcttgt

SEQ ID NO: 63 - DNA, 86 bp, Artificial Sequence
  Description of Artificial Sequence Random pool DNA molecules
acgtgtgcagctttcnnnnnnnnnnnnnnnnnnnnnnnnnnnnnnnnnnnnnnnnnnnnnnnnnnttattacggtaacgttggcac

SEQ ID NO: 64 - DNA, 18 bp, Artificial Sequence
  Description of Artificial Sequence Ligation template oligonucleotide
tgcacacgtagcgtcgct

SEQ ID NO: 65 - DNA, 15 bp, Artificial Sequence
  Description of Artificial Sequence PCR amplification primer 1.1
gtgccaacgttaccg

SEQ ID NO: 66 - DNA, 14 bp, Artificial Sequence
  Description of Artificial Sequence PCR amplification primer 2.29
acgtgtgcagcttc

SEQ ID NO: 67 - DNA, 15 bp, Artificial Sequence
  Description of Artificial Sequence PCR amplification primer 1.1r
gtgccaacgttaccg